(19) United States
(12) Patent Application Publication
(10) Pub. No.: US 2015/0005700 A1
(43) Pub. Date: Jan. 1, 2015

(54) SANITARY DEVICE FOR APPLYING AN ACTIVE PRINCIPLE WITHIN A BODY CAVITY

(71) Applicant: SOJASUN TECHNOLOGIES, NOYAL-SUR-VILAINE (FR)

(72) Inventors: Theo Efstathiou, Pace (FR); Christophe Morin, La Haie Traversaire (FR)

(73) Assignee: SOJASUN TECHNOLOGIES, NOYAL-SUR-VILAINE (FR)

(21) Appl. No.: 14/373,219

(22) PCT Filed: Jan. 16, 2013
(86) PCT No.: PCT/EP2013/050746

§ 371 Date: Aug. 28, 2014

(30) Foreign Application Priority Data
Jan. 19, 2012 (FR) 1250565

(51) Int. Cl. A61M 31/00; A61F 13/20

(52) U.S. Cl. CPC A61M 31/007; A61F 13/20; A61M 2210/0618; A61M 2210/1067; A61M 2210/1475
USPC 604/59

(57) ABSTRACT

The invention relates to a sanitary device (1) for applying at least one active principle inside a human or animal body cavity, said device comprising at least one biodegradable film (3) containing the at least one active substance and an applicator including: a main tubular body (4); and an ejector body (2) comprising a distal portion (202), at least one part of the surface of which forms a support for the at least one biodegradable film (3) and the end (203) of which is rounded, whereby said ejector body (2) can slide inside the main body (4) between at least one position in which the distal portion (202) of the ejector body (2) is located inside the main body (4) and an application position in which the distal portion (202) is located outside same, in the axial extension of the main body (4).

### 1. FIELD OF THE INVENTION

The field of the invention is that of sanitary devices used to apply an active principle or ingredient within a natural cavity of the human or animal body.

### 2. PRIOR ART

The treatment of certain conditions affecting natural body cavities, or the prevention of such conditions, can consist in administering an active ingredient systemically (in oral or injectable form) to the patient. However, this option has some drawbacks. In particular, the patient ingests a quantity of active ingredient that may be greater than necessary. This causes unnecessary exposure of the entire organism to these active ingredients which can be particularly harmful when they are chemical substances. Now, the ingesting of active ingredients, whether therapeutic or prophylactic, especially medicines, is not a trivial gesture. In addition to the effect of the dose taken, it is also necessary to take account of the negative effects on all the organs.

It is, for example, well known that the liver, which is the detoxifying organ for exogenous compounds, is slowly destroyed by medication over the long term. Thus, patients taking long-term treatment frequently develop hepatic pathologies such as medicine-related cirrhosis. The kidneys and the digestive system can also suffer substantially from chronic treatment. Finally, with regard to hormones, a very small quantity is enough to have an impact on the patient's entire organism (liver, brain, fat cells, genital and reproductive organs, etc).

One solution to overcome these drawbacks consists in locally delivering active ingredients used preventively or therapeutically by including them in compositions that take the form of pellets, gels, films, suppositories, foams, etc.

In gynecology, there is the known use of film to apply antibiotics, spermicides, anti-fungal products, etc to the surface of the vaginal mucus membranes.

However, such films have to be shaped in the form of tubes (for example by winding) so as to be introduced with the finger into the vagina. The result of this is that the treatment is shunned or very quickly halted by the patient herself at the very first signs of improvement. Now, adherence to a treatment is a major parameter in the success of the treatment. In addition, poor adherence to the treatment systematically leads to an increase in resistance to treatment on the part of bacteria, viruses and fungal infections.

There is therefore a need for a dedicated means for the simple and efficacious application of an active ingredient inside a natural cavity of the human body, especially the vagina, so as to improve the comfort of application and therefore improve adherence to the proposed treatment using such an active ingredient.

### 3. GOALS OF THE INVENTION

It is a goal of the invention therefore to provide a device enabling the local application of an active ingredient in a natural cavity of the human or animal body.

It is yet another goal of the invention, in at least one embodiment, to propose a device of this kind that is simple to use.

It is another goal of the invention, in at least one embodiment, to design a device of this kind, the manufacturing of which is simple and economical.

### 4. SUMMARY OF THE INVENTION

These goals as well as others that shall appear here below are achieved by means of a sanitary device for applying at least one active ingredient inside a human or animal natural cavity.

According to the invention, such a device comprises at least one biodegradable film containing at least one active ingredient and an applicator including:
a main tubular member having a first aperture at its proximal end and a second aperture at its distal end; and
an ejector member comprising a distal portion, at least one part of the surface of which forms a support for the at least one biodegradable film, and the end of which is rounded,
said ejector member being slidingly mobile within said main member between at least one position in which said distal portion of said ejector member is situated inside said main member and an application position in which said distal portion is situated outside the axial extension of said main member.

Thus, the invention relies on a wholly original approach proposing the use of an applicator integrating a piston-forming ejector member for the application, in a natural cavity, of a biodegradable film enclosing at least one active ingredient.

The term “biodegradable” is understood to mean that the film can be degraded naturally once it is applied inside a natural cavity of the human body in the conditions of temperature and humidity prevailing inside said cavity.

Through the simplicity of use of such an applicator, the user of the device according to the invention, the patient herself or the animal-care staff, is or are more likely to comply with the treatment protocol. Now, adherence to a treatment is a major element in its success.

It can be noted that the film could be provided independently of the applicator, the user then having to position the film on the applicator before using the device.

It will also be possible to plan for disposable devices in which the film will be already placed between the ejector member and the main member of the applicator. This positioning can be done by automation during the industrial-scale production of the device.

Such films are particularly suited to releasing relevant molecules into the organism. Preferably, these molecules are active pharmaceutical ingredients such as hormones, spermicides, contraceptives, antibiotics, anti-fungal products, anesthetic products etc. The targeted and local release of such active ingredients eliminates numerous harmful side effects of treatment administered systemically. Thus, the quantity of active ingredients to be incorporated into the formulation of such films is considerably diminished. At the same time, the same level of efficacy is maintained. The treatment therefore becomes less costly, for the consumer and/or for the public health organizations.

The biodegradable nature of such films also gives them many advantages. In particular, the degradation of the film is internal to the cavity and therefore does not produce waste material which could have to be withdrawn or eliminated.

Finally, the device according to the invention also lends itself to veterinary applications, for example for the treatment of veterinary pathologies for the improvement of fertility in a female of reproductive age.

Preferably, said ejector member and said main member each have an ellipsoid-shaped cross-section. Such an ellipsoid shape corresponds to a cylindrical shape of varying flatness of the ejector member and of the main member.

Such a shape makes it possible, as shall be seen more specifically here below in the present description, in a first stage, to shape the film accurately and easily when placing it in the applicator and, in a second stage, to unfold the film accurately without tearing or crumpling it inside the natural cavity in question. Thus, the use of such a device is considerably simplified for the user. Adherence to the treatment proposed by the composition can be appreciably improved.

In one advantageous embodiment, the biodegradable film preferably takes the form of a tongue, i.e. a thin, narrow and elongated shape. Such a tongue shape is especially suited to an ellipsoid-shaped section of the applicator. The user folds the tongue along its width on the ejector member of the applicator before inserting the entire unit into the main member. The advantages here are numerous: the direction along which the film is positioned on the applicator is unmistakable, no wrinkle is formed that could tear the film or create debris is made, and contact between the film and the user's finger is limited thus preventing contamination. The tongue shape of the film could also facilitate the automation of the production of disposable devices according to the invention.

Preferably, said tongue is essentially rectangular. This shape is very simple to produce on an industrial scale. The rectangular shape is also especially suited to the ellipsoid-shaped shape of the ejector member. In particular, it facilitates the placing of the film on the surface of the ejector member as well as its insertion into the main member without tearing or crumpling of the film. It also enables accurate unfolding of the film in the cavity.

In one particularly preferred embodiment, said tongue has a rectangular shape provided in its median part with at least two side notches. Thus, the widest parts of the tongue are placed on the surface of the ejector member while the median part having the notches covers the rounded end of the ejector member. The film is very simple to position and does not move when the unit is introduced into the main member.

Advantageously, the proximal end of the ejector member is provided with a first shoulder making it possible to stop the sliding of said ejector member inside said main member. The presence of a shoulder stops the travel of the ejector member in the main member and thereby makes it possible to control the motion of these different elements relative to each other. During the application of the film in the natural cavity, the user makes the ejector member on which the film is placed slide in the main tubular member until the film is made to completely come out of the main member. The presence of at least one shoulder blocks the progress of the ejector member and retains it in the main member.

Preferably, said tubular member furthermore comprises, on its distal end, at least two strips oriented towards the longitudinal axis of said device, said at least two strips being spaced out from one another and being curved inwardly towards the longitudinal axis of said tubular member and defining a central space. More preferably, the width of the orifice defined by the strips is equal to that of the film between the two notches. Thus, the film does not get torn during the releasing of the film. No film debris remains on the applicator device. The cleaning of the applicator is thereby facilitated and the user is sure that the dosage of active ingredient desired has been delivered. In addition, this characteristic makes it possible to design a device whose general shape is ergonomical. Thus, the introduction of the device according to the invention into a natural cavity is more comfortable and less painful. It furthermore limits any risk of injury.

In one advantageous embodiment, the dimensions of said main member are adjusted to those of the ejector member. This prevents any deformation of the film between these two elements.

Preferably, said ejector member comprises a distal portion and a proximal portion separated by a second shoulder, said distal portion being intended to receive said biodegradable film. The presence of a second shoulder demarcating the distal portion informs the user when and how to position the film on the applicator. In addition, this shoulder maintains the film in position and prevents it from sliding along the ejector member when it is introduced into the main member. Finally, it is particularly useful, when fitting together the ejector member with the film and then the main member, to be able to block the ejector member inside the main member. This makes it possible not to have to maintain it while the device is introduced into the body. The grasping and application of the film is thereby particularly facilitated for the user.

In one preferred variant, said proximal portion has a cross-section greater than the cross-section of said distal portion.

Advantageously, said ejector member has at least one reinforcement piece extending at least partly along a lateral length of said ejector member. Such reinforcement pieces make it easier to guide the ejector member when it is introduced into the main member. They furthermore contribute to holding the film in position, preventing it from slipping on the sides and being subjected to a twisting motion when the ejector member and the film are introduced into the main member. This therefore prevents, among other inconvenient features, the tearing of the film in the applicator.

According to one embodiment, said main member furthermore comprises means at its proximal end facilitating the grasping of said device.

Preferably, the external surface of said main member is at least partly provided with relief features. Such means make it easy to take hold of the device and simplify the application of the film in the cavity. Thus the gesture of application is simple, swift and reliable. The action soon becomes automatic for the user and treatment then becomes a routine gesture which facilitates and improves adherence to the treatment.

The applicator of the device according to the invention will preferably be made out of a non-allergenic plastic material resistant to sterilization such as polyethylene, polypropylene, polyethylene teraphtalate, etc. This material can especially be advantageously itself biodegradable and can be chosen among starch-based polymers, polyhydroxyalkanoate (PHA), polyhydroxybutyrate (PHB), polyhydroxyvalerate (PHV), polylactic acid (PLA), a cellulose-based polymer, chitosan, a polypeptide, polycaprolactone (PLC), polyester amide (PEA), aliphatic copolyester (PBSA) and the members of the polyester family. This variant is particularly useful when the device is a disposable device.

The device of the invention makes it possible to deposit a film in a natural cavity, especially the vaginal cavity, as well as the rectal or nasal cavity. The dimensions of the device according to the invention will be determined according to its use and the nature of this cavity.

For a variant in which the device according to the invention is intended for use in animals, the ejector member has a length of 30 mm to 900 mm, a greatest diameter of 3 mm to 200 mm and a smallest diameter of 2 mm to 190 mm. In this variant, the main member has a length of 15 mm to 600 mm, a greatest diameter of 4 mm to 201 mm, and a smallest diameter of 3 mm to 191 mm. According to another variant, when the device according to the invention is intended for human application, the ejector member has a length of 30 mm to 200 mm, a greatest diameter of 3 mm to 50 mm and a smallest diameter of 2 mm to 49 mm. In this variant, the main member has a length of 15 mm to 150 mm, a greatest diameter of 4 mm to 51 mm and a smallest diameter of 3 mm to 50 mm.

These dimensions make it possible to provide the device according to the invention with various uses in humans, especially nasal, vaginal, anal/rectal or mouth applications. These dimensions are moreover perfectly suited for use in animals such as cattle, sheep and horses, etc.

### 5. LIST OF FIGURES

Other features and advantages of the invention shall appear more clearly from the following description of an embodiment intended for the application of a biodegradable film in the vagina, given by way of a simple illustratory and non-exhaustive example and from the appended drawings, of which:

FIG. 1 is an exploded view in perspective of this embodiment;

FIG. 2 is a longitudinal sectional view of the device according to the invention in an assembled position;

FIG. 3 is a longitudinal sectional view of the device according to the invention in the position of application;

FIG. 4 is a longitudinal sectional view of the ejector member of the device according to the FIG. 1;

FIG. 5 represents the pattern of the biodegradable film of the device according to the invention;

FIG. 6 is a longitudinal sectional view of the main member of the device according to FIG. 1.

### 6. DESCRIPTION OF ONE EMBODIMENT OF THE INVENTION

The general principle of the invention relies on the designing of a device comprising a biodegradable film including at least one active ingredient and one applicator comprising an ejector member and a main member. The two members forming the applicator preferably have ellipsoid-shaped cross-sections and the film is preferably tongue-shaped. These sections and shapes enable the film to be placed easily on the flattened faces of the ejector member. In one valuable variant, the applicator according to the invention is made out of a plastic material, preferably derived from biodegradable, “green” chemistry. Thus, the entire device is more compliant with the environment.

### 6.1. Applicator of a Biodegradable Film for Gynecological Indications

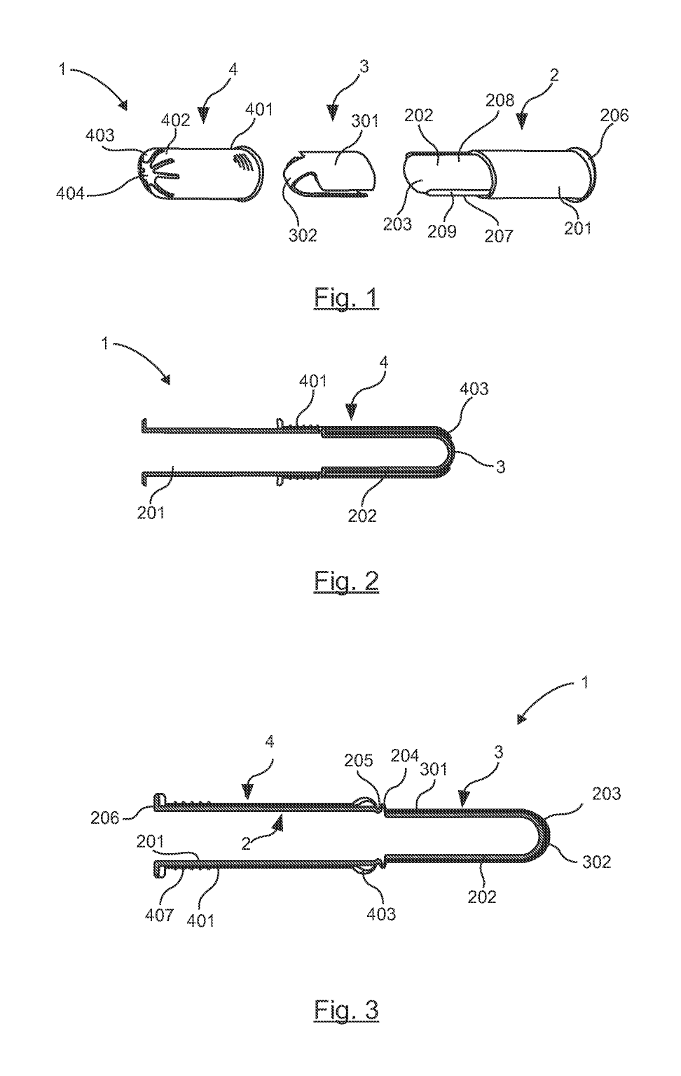

Referring to FIG. 1, we present an exploded view and a view in perspective of an embodiment of the device according to the invention.

The sanitary device 1 comprises an applicator and a film. The applicator is made out of plastic material chosen from among polyethylene, polypropylene and polyethylene teraphtalate. The applicator comprises an ejector member 2 with an ellipsoid cross-section, a biodegradable film 3 and a main member 4 with an ellipsoid-shaped cross-section.

The biodegradable film 3 covers the ejector member 2 and the unit thus formed is then introduced into the proximal part 401 of the main member 4.

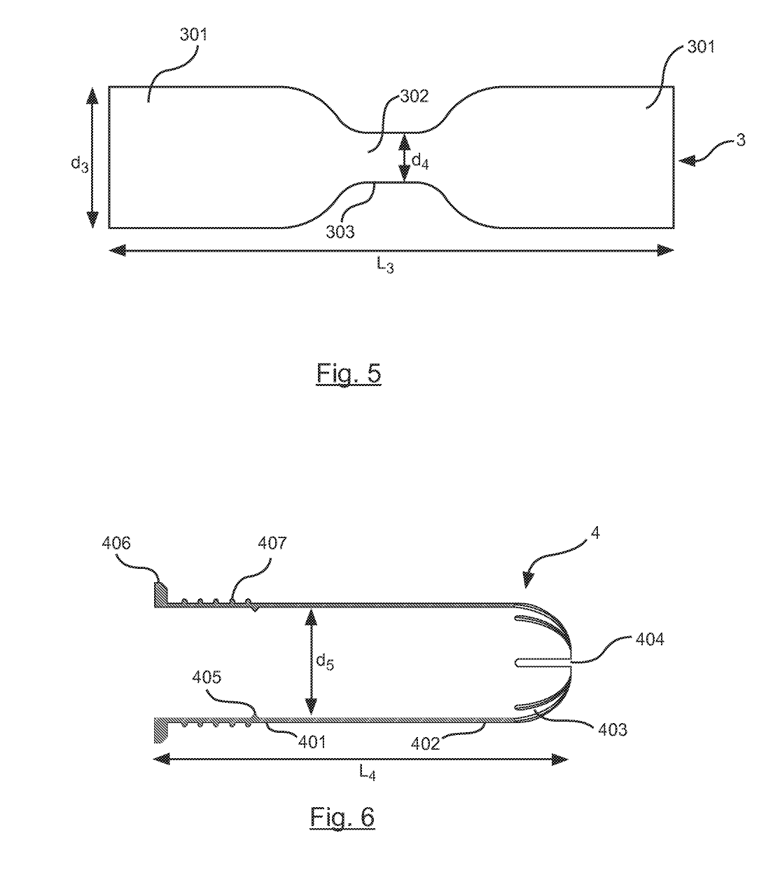

The size of the film is adapted to that of the applicator. In particular, referring to FIG. 5, the film 3 in this embodiment is a tongue which is substantially rectangular and comprises two notches or indentations 303 in the median part 302. The film is folded at the level of the median part 302, of smaller width, to cover the ejector member 2. More specifically, the wide edges 301 of the tongue 3 cover the upper face 208 and lower face 207 of the ejector member while the part of smaller width 302 covers the distal end 203 of the ejector member 2. The ejector member, in this embodiment, has lateral reinforcements 209 and an annular shoulder 204 at its median part. The lateral reinforcements 209 herein take the form of a shoulder extending throughout the lateral length of the distal part 202 of the ejector member 2. These different elements especially have the following utility:
guiding the ejector member 2 when it slides in the main member 4, and
stabilizing the film 3 when it is situated on the ejector member in order to prevent it from slipping or shifting or twisting during the insertion of the unit formed by the member 2 and the film 3 into the main member 4.

The main member 4 is provided, at its distal end, with a set of flexible strips 403 defining an orifice 404. Referring to FIGS. 1 and 5, the width d4 of the film between the notches is equal to or smaller than that of the orifice 403. Similarly, the width d3 of the film 3 at its wide edges 301 is appreciably equal to the greatest diameter of the ellipsoid-shaped section of the ejector member 2.

FIG. 2 is a longitudinal section of the device according to FIG. 1 in an assembled position when the user carries out the assembling of the device or when she procures the already assembled device. It can be seen that the distal portion 202 of the ejector member is covered with the film 3, the entire unit being introduced into the main member 4. When the film is being applied, the user exerts a thrust on the proximal part of the member 2 in order to make it slide within the member 4. The device is provided with locking means to stop the sliding of the ejector member in the main member.

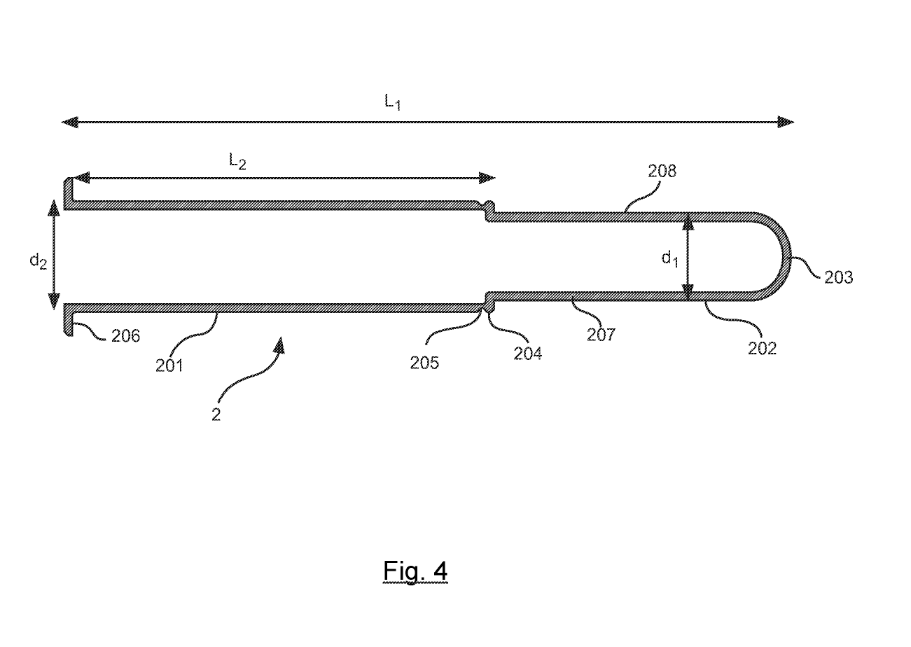

As can be seen in FIG. 3, when the device is in the application position, the proximal portion 201 of the ejector member 2 is inside the main member 4. The distal portion 202 on which the film 3 is positioned is then entirely out of the main member 4. The film 3, which is just laid on and does not adhere to the ejector member 2 gets detached from it naturally and remains in the natural cavity. The active ingredient that it contains can then be released into the organism.

A more precise view of the ejector member is shown in FIG. 4. The ejector member 2 is tubular and hollow and has an ellipsoid-shaped cross-section defined by a greater diameter and a smaller diameter. The greater diameter which corresponds to the width of the ejector member 2 is 20 mm in this embodiment. The member 2, in its median part has an annular shoulder 204 which demarcates, in the member 2, a proximal portion 201 as well as a distal portion 202. The end 203 of the distal portion 202 is rounded for greater ergonomy. The ejector member 2 has a wall, the thickness of which is about 1 mm. The length of the proximal portion 201 is 54 mm and its smallest diameter d2 is equal to 14 mm. The smallest diameter dl of the distal portion 202 is equal to 11 mm. The annular shoulder 204 is adjacent to an annular groove 205 which is to cooperate with the main member 4. The total length L1 of the ejector member 2 is equal to 92 mm while the length L2 of the proximal portion is equal to 54 mm. The member 2 furthermore has, at its proximal end, a shoulder 206. This shoulder especially makes it easier for the user to grasp the member when she handles the device according to the invention and especially when she applies the film into a cavity of her organism.

Referring to FIG. 5, we present a pattern of the biodegradable film 3. This film takes the form of a tongue. This film has at least one therapeutic active ingredient, for example an anti-fungal substance, an antibiotic, a spermicidal agent etc. The film 3 is substantially rectangular, comprising two notches 303 in its median part 302, i.e. its general shape is within a rectangle with a width d3 and a length L3. The total length L3 of the film is about 80 mm. The thickness of the film is approximately d′ 1 mm. The width d3 of the edges 301 is equal to 20 mm, i.e. it is appreciably equal to the largest diameter of the ellipsoid-shaped section of the ejector member. The width between the notches d4 is about 7 mm. This tongue is measured between the parallel sides of the notches.

FIG. 6 shows a side view of the main member 4. In this embodiment, the main member has a total length L4 of 53 mm and a diameter d5 of 14 mm. The main member has an aperture at its proximal end enabling the insertion of the ejector member 2 covered with film 3 during the assembling of the device as well as an orifice 404 at its distal end 402 enabling the application of the film 3. As indicated here above, this main member is provided with a plurality of flexible strips 402. These strips define the orifice 404 at their apex. They are also spaced out relative to each other, enabling their relative motion during the sliding of the member 2 in the member 4. Their flexibility is obtained especially through a smaller thickness relative to the thickness of the proximal portion 401 and distal portion 402 of the main member 4. This flexibility of the strips enables the ejector member supporting the film to be easily brought out without tearing or crumpling or without retention of a film inside the main member. Thus, the application of the biodegradable film for gynecological use is particularly simplified. The member 4 furthermore has a relief part 407 on the external surface of its proximal portion 401. This relief part 407 prevents any sliding or slipping of the device between the user's hands. The gesture is therefore more reliable, faster, simpler and more precise. The user therefore has no apprehension about her treatment nor any resistance to it.

Another advantage of the device according to the invention is the presence of means for blocking the relative motion of the ejector member 2 in the main member 4. These means comprise especially the presence of shoulders at the proximal ends of the ejector member 2 and the main member 4. In this embodiment, the shoulder of said main member abuts the shoulder 206 of the ejector member when the device is in its application position. The travel of the ejector member inside the main member is then controlled by mechanical means. In addition, the main member 4 can have a shoulder 405 in its proximal part 401. This shoulder 405 is intended for cooperating with the annular groove 205 of the ejector member 2 when the device according to the invention is assembled before application of the film. Thus, the main member 4 is held around the member 2 and the film 3 before any use of the device according to the invention. This protects the film before it is applied.

This blocking of the mechanical nature is however not irreversible. A slight pressure exerted by the user on the shoulder 206 of the ejector member 2 releases the shoulder 405 from the annular groove 205 and makes the ejector member 2 slide in the main member 4.

### 6.2. Variants

The ejector member according to the invention can take the form of a piston. The distal part is then replaced by a rod terminated by a plunger wide enough to abut the proximal shoulder 406 of the main member.

The device according to the invention can also be used for rectal applications, both among children and adults. In this case, the film can contain different substances that have to be rapidly absorbed by the organism, for example nausea-quelling substances.

Nasal applications are also useful for the relieving sinus congestion. Indeed, the efficacy of drops and sprays is often limited because the liquid flows and does not remain sufficiently in contact with the nasal lining Furthermore, the application of a film resolves problems related to the often excessive tonicity of nasal solutions.

An applicator device according to the invention can also be useful for treating cases of mild or severe epitaxis (nose-bleeds). The administration of a nasal spray can sometimes be enough to stop mild nose-bleeds. However, the preferred treatment in the case of major bleeding is nasal packing. There are two methods of nasal plugging:
anterior nasal packing: the pack is introduced into the nostril; and
anterior nasal packing: the pack is introduced in the back of the throat and is then removed. This removal of the pack can sometimes cause irritation, leading to a recurrence of the epitaxix.

The use of biodegradable films containing vasoconstrictive substances therefore makes it possible to stop nose-bleeds whatever their size. In addition, because of their biodegradable nature, the films break up spontaneously and therefore prevent the inconvenience related to the removal of a pack. Examples of vasoconstrictive substances that can be contained in such films are bicarbonates, angiotensin II, adrenaline, noradrenaline, anti-diuretic hormones, endotheline, etc. The biodegradable films can also contain healing substances to heal wounds more rapidly and prevent recurrence.

Another example of a use of the device according to the invention is that of a mouth applicator for dental-stomatological treatment. The films deposited by means of such applicators may contain:
anesthetics for local anesthesia preceding medical operations; or
vasoconstrictive substances to stop bleeding.

Thus, the pain entailed in the injection of a local anesthetic into an inflamed gum is avoided. The risk of infection related to the penetration of the needle into the gum is also eliminated. Furthermore, when the gum bleeds during treatment, the practitioner chooses either to draw out the blood or to place a pack to absorb and plug the wound. The application of a vasoconstrictive film and/or healing film on the surface of the wound on the gum swiftly stops bleeding.

The device according to the invention is also suitable for the application of a film for anal/rectal purposes. Such a device can be used to prevent or treat pathologies such as hemorrhoids, anal fissures, abscesses, discharges, infections, sexually transmissible diseases, etc. When the film contains chemotherapy substances, the device according to the invention can be used for the targeted and local treatment of anal/rectal tumors. Thus, the treatment of the tumor gains in efficacy while limiting the negative effects of chemotherapy on the patient's entire organism.

The device according to the invention is also suited to veterinary treatment and care, especially for the intra-vaginal application of sexual hormones to improve or treat a problem of fertility.

The dimensions of the device according to the invention can be adapted according to the anatomical destination, the patient's size and age, using general knowledge known to those skilled in the art.

1. Sanitary device (1) for applying at least one active ingredient inside a human or animal natural cavity, said device comprising at least one biodegradable film (3) containing at least one active ingredient and an applicator including:
a main tubular member (4) having a first aperture at its proximal end (401) and a second aperture at its distal end (402); and
an ejector member (2) comprising a distal portion (202), at least one part of the surface of which forms a support for the at least one biodegradable film (3), and the end (203) of which is rounded,
said ejector member (2) being slidingly mobile within said main member (4) between at least one position in which said distal portion (202) of said ejector member (2) is situated inside said main member (4) and an application position in which said distal portion (202) is situated outside the axial extension of said main member (4).

2. Device according to claim 1 characterized in that said ejector member (2) and said main member (4) each have an ellipsoid-shaped cross-section.

3. Device according to claim 1 characterized in that said biodegradable film (3) takes the form of a tongue.

4. Device according to claim 3 characterized in that said tongue has an essentially rectangular shape.

5. Device according to claim 4 characterized in that said tongue has a rectangular shape provided in its median part (202) with at least two side notches (303).

6. Device according to claim 1 wherein the proximal end (201) of said ejector member (2) is provided with a first shoulder (206) making it possible to stop the sliding of said ejector member (2) inside said main member (4).

7. Device according to claim 1 wherein said tubular member (4) furthermore comprises, on its distal end (402), at least two strips (403) oriented towards the longitudinal axis of said device, said at least two strips being spaced out from one another and being curved inwardly towards the longitudinal axis of said tubular member, defining a central space (404).

8. Device according to claim 1 wherein the dimensions of said main member (4) are adjusted to those of said ejector member (2).

9. Device according to claim 1 wherein said ejector member (2) comprises a distal portion (202) and a proximal portion (201) separated by a second shoulder (204), said distal portion (202) being intended to receive said biodegradable film (3).

10. Device according to claim 9, characterized in that said proximal portion (201) has a cross-section greater than the cross-section of said distal portion (202).

11. Device according to claim 1 wherein said ejector member (2) comprises at least one reinforcement piece (206) extending at least partly along a lateral length of said ejector member (2).

12. Device according to claim 1 wherein said main member (4) furthermore comprises means at its proximal end (401) facilitating the grasping of said device f.

13. Device according to claim 1 according to which the external surface of said main member is at least partly provided with relief features (407).

14. Device according to claim 1 characterized in that said applicator is constituted by a non-allergenic plastic material resistant to sterilization such as polyethylene, polypropylene, polyethylene teraphtalate, a arch-based polymer, chitosan, polyhydroxyalkanoate (PHA), polyhydroxyvalerate (PHV), polyhydroxybutyrate (PHB), polylactic acid (PLA), a cellulose-based polymer, a polypeptide, polycaprolactone (PLC), polyester amide (PEA), aliphatic copolyester (PBSA) and the members of the polyester family.
